(19) United States
(12) Patent Application Publication
(10) Pub. No.: US 2009/0234198 A1
(43) Pub. Date: Sep. 17, 2009

(54) HEALTHCARE KNOWLEDGEBASE

(76) Inventor: Kimberly Vorse, Ketchum, ID (US)

Correspondence Address: Zarian Midgley & Johnson PLLC, University Plaza, 960 Broadway Ave., Suite 250, Boise, ID, 83706, US

(21) Appl. No.: 12/123,292

(22) Filed: May 19, 2008

Related U.S. Application Data
(60) Provisional application No. 61/036,441, filed on Mar. 13, 2008.

(51) Int. Cl. A61B 5/00

(52) U.S. Cl. 600/301

(57) ABSTRACT

A method is provided for automatically retrieving information from a computerized healthcare knowledgebase. The method involves collecting data about a user from one or more biometric sensors, analyzing the data to determine a condition of the user, processing the user condition information to formulate a query of the computerized healthcare knowledgebase for relevant content, and presenting the relevant content to the user in an automated fashion. The healthcare knowledgebase includes a plurality of records having a reference to healthcare related multimedia content and numeric biometric limit parameters indicating one or more physiological conditions to which the referenced content is relevant. The numeric biometric limit parameters may include rolling statistical information that is updated periodically based on data collected from a plurality of users over time.

### CROSS-REFERENCE TO RELATED APPLICATIONS

This application claims the benefit under 35 U.S.C. §119(e) of U.S. Provisional Patent Application No. 61/036,441 filed Mar. 13, 2008, entitled HOME POLYSOMNOGRAM, the disclosure of which is hereby incorporated herein by reference in its entirety.

### BACKGROUND

The present application relates generally to automated healthcare systems and, more specifically, to systems with biometric sensors that collect data from a patient and utilize the collected data to assist with the diagnosis and treatment of the patient in an automated fashion.

There is a growing strain on the healthcare system due in part to a shortage of doctors and other qualified healthcare professionals, especially in poor and rural areas. The most common mode of delivering healthcare information and treatment is through personal, face-to-face contact between a healthcare provider and a patient. But due to the growing number of patients to treat and the limited time available to dedicate to each patient, in addition to the ever expanding body of knowledge regarding new drugs and treatments, something has to give.

Although traditional techniques for disseminating healthcare information and treatments are generally accurate, it can be appreciated that the expense and time associated with these traditional techniques results in increased costs and inconvenience on the part of the patient. Such conventional approaches are often time-consuming and cannot be employed at the convenience of the patient. Many patients are interested in maintaining good health and would get regular health checkups if they were not so expensive and time-consuming. Traditional healthcare techniques frequently result in delaying diagnosis at the expense of preventative medicine. Also, there is a risk to the patient of inaccurate self-diagnosis and treatment.

One attempt to alleviate this problem has emerged in the form of “telemedicine,” which enables healthcare professionals to provide interactive healthcare to patients remotely, utilizing technology and telecommunications. In some cases, telemedicine allows patients to interact with physicians in real-time over long distances, sometimes via video conferencing. In other cases, telemedicine involves capturing patient data, such as video or still images, storing the data, and transmitting it to physicians for diagnosis and follow-up treatment at a later time. While telemedicine has certainly helped to address the growing shortage of doctors, unfortunately our healthcare system is still stretched, leaving many to access medical care only after serious complications arise.

### SUMMARY

The above-mentioned drawbacks associated with existing healthcare systems are addressed by embodiments of the present application, which will be understood by reading and studying the following specification

In one embodiment a method is provided for automatically retrieving information from a computerized healthcare knowledgebase. The method comprises collecting data about a user from one or more biometric sensors, and analyzing the data to determine a condition of the user. The method further comprises processing the user condition information to formulate a query of the computerized healthcare knowledgebase for relevant content, and presenting the relevant content to the user in an automated fashion.

008 In another embodiment, a healthcare knowledgebase includes a plurality of records. Each record comprises a reference to healthcare related multimedia content and numeric biometric limit parameters indicating one or more physiological conditions to which the referenced content is relevant. The numeric biometric limit parameters comprise rolling statistical information that is updated periodically based on data collected from a plurality of users over time.

In another embodiment, an automated system presents healthcare information to a patient. The system comprises a patient data module with one or more biometric sensors for collecting data from the patient and a compute module in communication with the patient data module, the compute module being configured to analyze the data collected by the biometric sensors by referencing a symptom database. The system further comprises a query module in communication with the compute module, the query module being configured to query a healthcare knowledgebase for information relevant to a condition of the patient, based on the data collected by the biometric sensors. The healthcare knowledgebase comprises a plurality of multimedia presentations and articles.

These and other embodiments of the present application will be discussed more fully in the description. The features, functions, and advantages can be achieved independently in various embodiments of the claimed invention, or may be combined in yet other embodiments.

### BRIEF DESCRIPTION OF THE DRAWINGS

FIG. 1 is a block diagram of a system for collecting biometric sensor data for analysis and diagnosis of various ailments to generate targeted, user-specific reports.

FIG. 2 is a more detailed diagram of the system shown in FIG. 1.

FIG. 3 shows one embodiment of a user diagnosis.

FIG. 4 illustrates a table that may be included in one possible embodiment of a healthcare knowledgebase.

FIG. 5 is a flow chart illustrating a method for retrieving information from a healthcare knowledgebase.

### DETAILED DESCRIPTION

In the following description, reference is made to the accompanying drawings that form a part thereof, and in which is shown by way of illustration specific exemplary embodiments in which the invention may be practiced. These embodiments are described in sufficient detail to enable those skilled in the art to practice the invention, and it is to be understood that modifications to the various disclosed embodiments may be made, and other embodiments may be utilized, without departing from the spirit and scope of the present invention. The following detailed description is, therefore, not to be taken in a limiting sense.

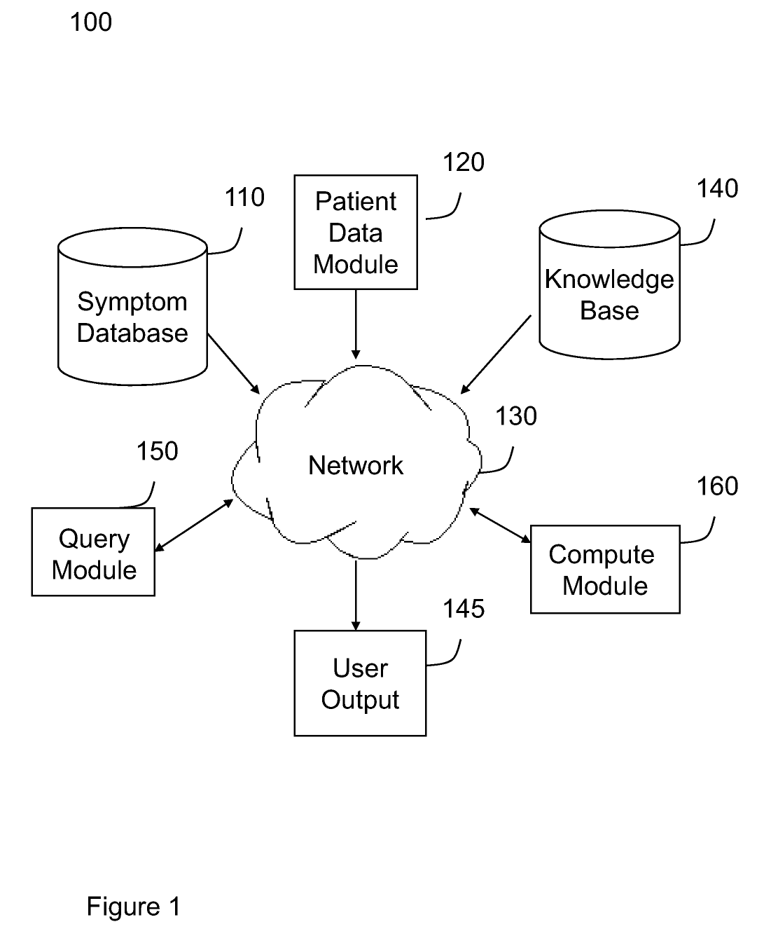

FIG. 1 is a block diagram showing one embodiment of a system 100 for collecting biometric sensor data from a patient for subsequent analysis. As described in more detail below, the system 100 analyzes the data collected by the biometric sensors and utilizes the data to retrieve information from a healthcare knowledgebase with content that is meaningful and beneficial to patients having little or no healthcare training or expertise. As a result, the system 100 can advantageously provide patients with customized information that is targeted to their specific health conditions, as determined by the biometric sensors.

As described below, the system 100 is preferably designed to be used directly by patients, without the need for a prescription or other intervention by a doctor or any other trained healthcare worker. Accordingly, it is envisioned that the system 100 can be utilized to disseminate healthcare information and treatments to large segments of the population efficiently and in a customized and targeted manner, that cannot be accomplished using traditional approaches to healthcare diagnosis and treatment.

In the embodiment illustrated in FIG. 1, the system 100 comprises a symptom database 110, a patient data module 120, a knowledgebase 140, a query module 150, and a compute module 160, all connected via a suitable network 130. The network 130 may comprise public or private busses for internal communication within a common hardware chassis, or a telecommunications network, such as a LAN, WAN, or the Internet, for transmitting data between connected nodes or elements.

In some embodiments, the compute module 160 may comprise microprocessors, signal conditioning devices and various algorithms. The patient data module 120 may comprise sensors and signal conditioning devices used to collect and output user data. The compute module 160 may use the output of the patient data module 120 as an input for analysis. The compute module 160 may access, via the network 130, the symptom database 110 comprising data regarding human ailments and conditions. The compute module 160 may also access the query module 150 and knowledgebase 140 via the network 130.

In some embodiments, the query module 150 comprises algorithms and software or hardware methods to obtain appropriate data from the knowledgebase 140. As described in more detail below, the knowledgebase 140 may comprise a variety of material relating to human health information. Utilizing the symptom database 110, the patient data module 120, the query module 150, and the knowledgebase 140, the compute module 160 may generate user output 145. The user output 145 may comprise educational material, multimedia material, and treatment plans that are targeted, meaningful, and relevant to the user.

In operation, the compute module 160 receives input from the patient data module 120 and the symptom database 110, via the network 130, to generate one or more inputs for the query module 150. The query module 150, in turn, may use these inputs to query the knowledgebase 140 via the network 130. The output of the query module 150 may then be used by the compute module 160 to generate user output 145. Accordingly, the system 100 advantageously enables the user to be presented with customized specific output 145 that is unique to the user's needs and conditions. For example, in some embodiments, the output 145 may include pertinent articles authored by reliable experts, relevant videos or other multimedia experiences, or targeted professional referrals and recommendations.

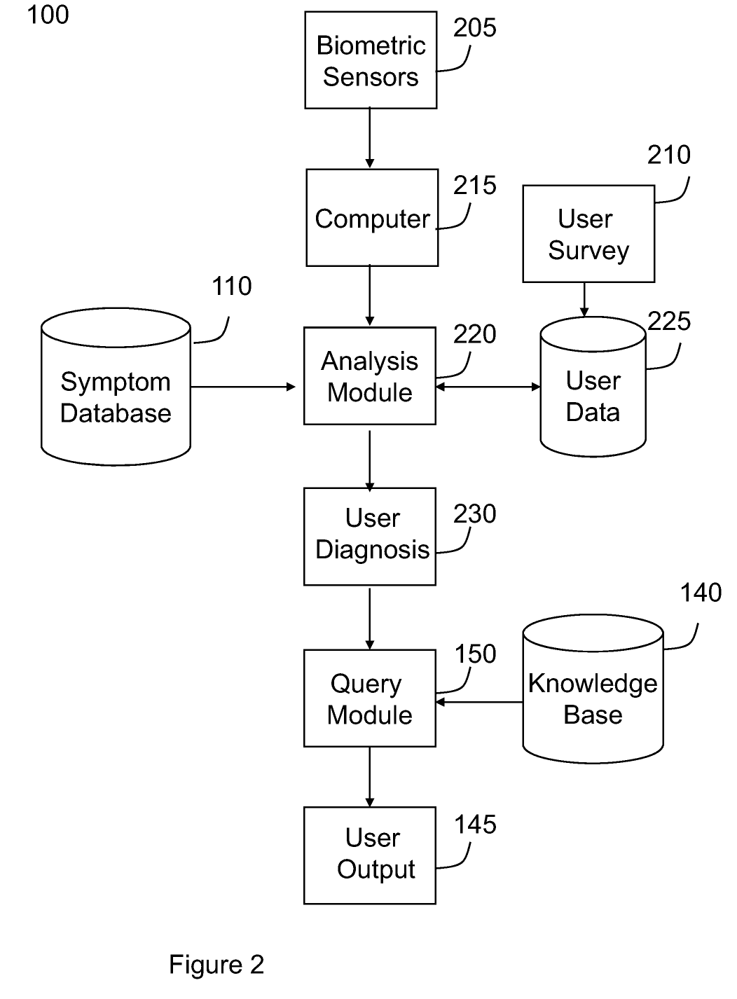

FIG. 2 is a more detailed diagram of the system 100, shown in FIG. 1. In the embodiment illustrated in FIG. 2, the system 100 comprises one or more biometric sensors 205 coupled to a computer 215. The biometric sensors 205 may comprise a wide variety of suitable sensors such as, for example, EKGs, EEGs, blood pressure monitors, oxygen saturation sensors, motion sensors, photodiodes, light sensors, microphones, REM sensors, respiratory monitors, glucose sensors, or blood analysis devices. The biometric sensors 205 collect biometric data from a user, which can be interpreted or processed by the biometric sensors 205, or transmitted as raw data to the computer 215. The computer 215 may comprise a wide variety of computing devices, such as, for example, a desktop computer, notebook computer, tablet computer, handheld computer, PDA, mobile phone, or the like. While in the illustrated embodiment, the biometric sensors 205 and the computer 215 as shown as separate components, those of ordinary skill in the art will understand that in some cases the computer 215 is built into the biometric sensors 205.

The computer 215 may condition the output of the biometric sensors 205, and provide the conditioned data as input for the analysis module 220. In the illustrated embodiment, the analysis module 220 is coupled to a symptom database 110 and user data 225. As described above, the symptom database 110 may comprise expert systems, proprietary databases or commercial databases. The user data 225 may comprise personal data such as age, weight, height, or past biometric sensing data. In some cases, the user data 225 may include the output of an optional user survey 210. The user survey 210 may gather a variety of pertinent information such as user behavior, blood sugar levels, diet, previous medical history, and family history.

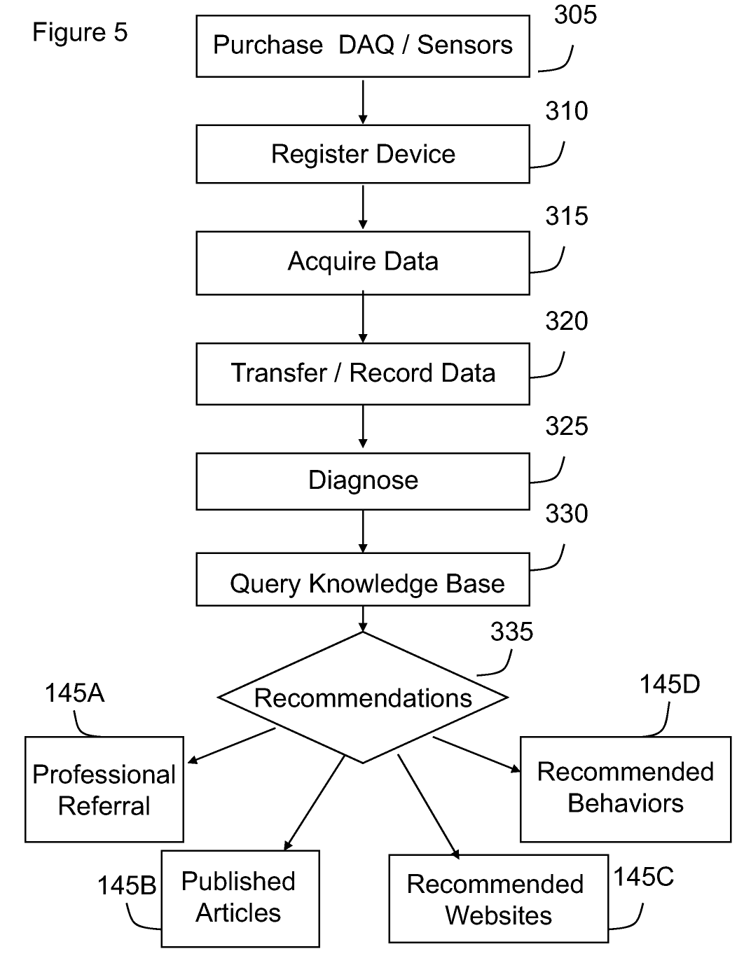

The analysis module 220, perhaps with the assistance of the compute module 215, may interpret the data from the biometric sensors 205 and the user data 225, perform mathematical analysis, and generate statistics that could be utilized and compared against the symptom database 110. Thus, the analysis module 220 advantageously abridges and summarizes the raw data or conditioned data received from the computer 215 to generate a more useful output in the form of a user diagnosis 230, such as the sample user diagnosis 230 shown in FIG. 5.

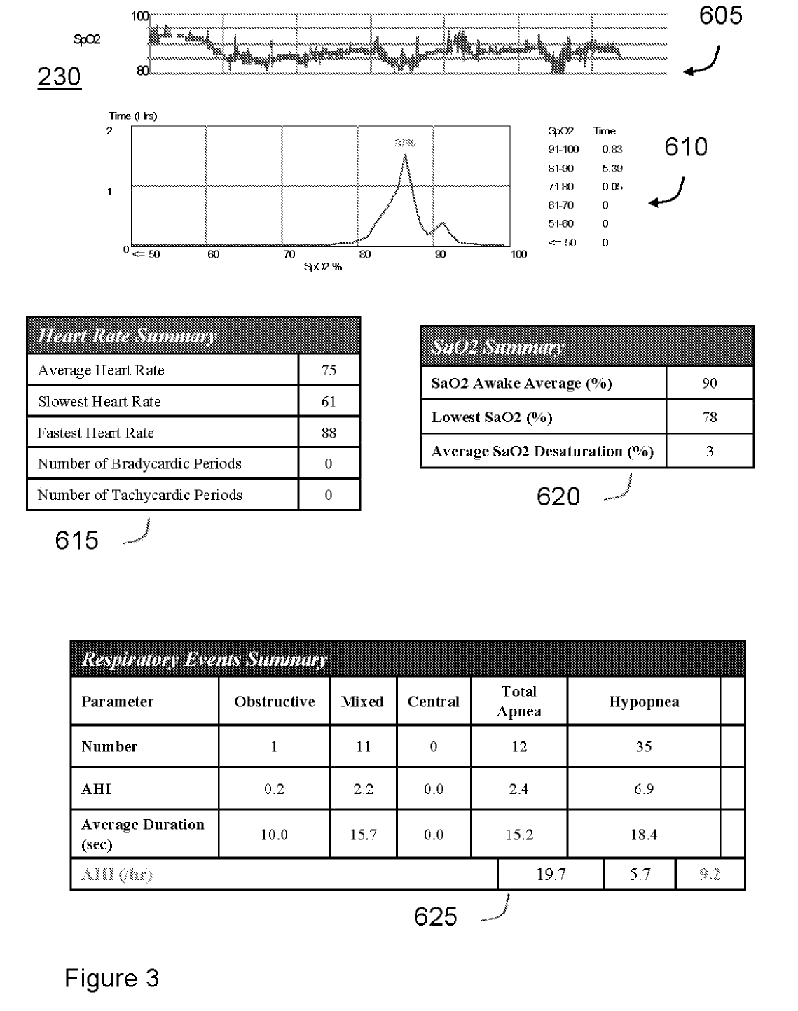

In some embodiments, the user diagnosis 230 comprises graphs, charts, biometric parameters, and possibly a list of conditions. For example, in the embodiment shown in FIG. 3, the user diagnosis 230 includes biometric parameter charts such as a temporal oximetry tracing 605 and a time versus O₂ saturation graph 610, in addition to a heart rate summary table 615, SaO₂ summary table 620, and a respiratory events summary table 625.

While the user diagnosis 230 generated by the analysis module 220 is often useful to a physician or other trained healthcare worker, it frequently has only limited value to a user without healthcare training or expertise. Therefore in the system 100, the user diagnosis 230 is provided as an input to the query module 150, which is also coupled to the knowledgebase 140. As described in more detail below, the knowledgebase 140 contains content that is designed to be useful to a patient with little or no healthcare training, such as published articles, instructional videos, treatment plans, behavior modification techniques, websites, professional directories, and proprietary data.

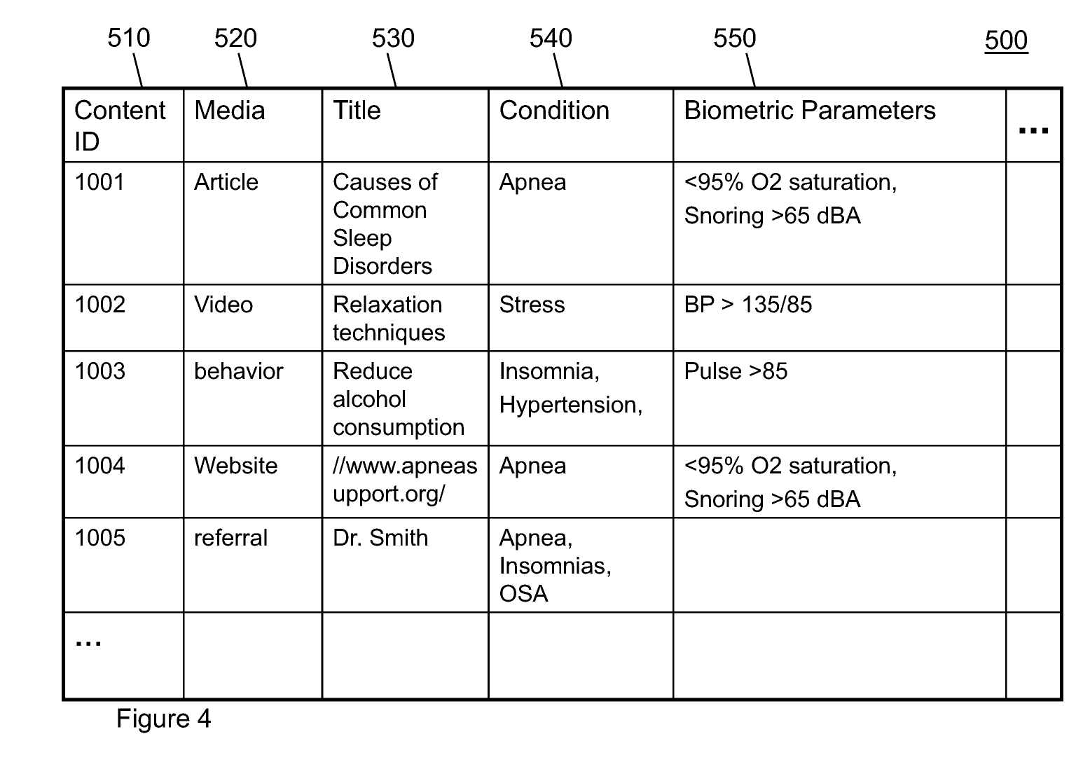

FIG. 4 illustrates a table 500 that may be included in one possible embodiment of the knowledgebase 140. The sample table 500 is intentionally simplified to provide brevity and clarity. In this particular example, the knowledgebase 140 comprises a simple flat file database containing records with a series of fields, such as a content ID field 510, media type field 520, title field 530, condition field 540, and biometric parameters field 550, among others. The content ID field 510 provides a unique identifier associated with each record in the table 500.

The media type field 520 indicates what type of content or data each record contains. As shown in FIG. 4, the knowledgebase 140 may contain a variety of suitable types of media, such as articles, multimedia presentations (e.g., audio or video podcasts, animations, or photographs), links to appropriate websites, referrals to medical personnel such as sleep disorder specialists, physical therapists, or dieticians, or recommended behaviors, such as exercises, sleep positions, or eating suggestions.

In the illustrated embodiment, the table 500 includes a title field 530, indicating the title of the corresponding record. In other embodiments, the table 500 may also comprise a plurality of similar fields listing additional bibliographic information about each record, such as author, date, length, public or proprietary, sensitive or graphic, age appropriateness, and others. The condition field 540 comprises conditions or diagnoses to which the corresponding record is applicable. The conditions may be general in nature, such as “stress related,” or more specific, such as “mild obstructive sleep apnea.” The biometric parameters field 550 may contain biometric thresholds, ranges or limits, indicating one or more patient conditions to which the corresponding record applies. For example, the biometric parameters field 550 may comprise thresholds, such as “pulse rate greater than 85 beats per minute” or “oxygen saturation less than 92%, blood glucose greater than 126 mg/dl.”

Referring again to FIG. 2, the query module 150 may reference the user diagnosis 230 to formulate one or more queries to retrieve information from the knowledgebase 140. As described below, such queries may take a variety of forms, such as, for example, simple look-up table queries, proprietary algorithms, relational structured queries, or artificial intelligence. For example, the sample table 500 shown in FIG. 4 supports a simple table look-up query method of retrieving information from the knowledgebase 140. One example of a simple query may be to find records having a condition field 540 containing the phrase “mild apnea.”

The embodiment illustrated in FIG. 4 also supports a query method implementing Boolean logic, such as finding records having a biometric parameters field 550 indicating a blood pressure greater than 120/80 AND a pulse greater than 75 beats per minute. Those of ordinary skill in the art will understand that, in other embodiments, the knowledgebase 140 may comprise structured data tables, relational data sets, or free form data and the corresponding query methods may involve complex algorithms, fuzzy logic or artificial intelligence, among others.

Referring again to FIG. 2, after the query module 150 queries the knowledgebase 140, it generates the user output 145. In some embodiments, the user output 145 comprises specific content of interest to the user based upon the user's unique conditions and circumstances, as determined by the biometric sensors 205. For example, the user output 145 may comprise videos demonstrating relaxation techniques, articles on common causes of mild obstructive sleep apnea, recommendations to reduce caffeine and alcohol consumption, or recommendations of specific medications or products, such as Continuous Positive Airway Pressure (CPAP) devices or Breathe Right® nasal strips.

The user output 145 can also provide rolling statistics regarding the effectiveness of prior recommendations, based on data collected from a plurality of users. For example, the user output 145 may provide feedback such as “63% of consumers with mild sleep apnea similar to yours reported a ‘great’ improvement in their sleep quality with use of Breathe Right® nasal strips, and there was a 45% reduction in arousals in when this subgroup used Breathe Right® nasal strips.” Such rolling statistical information can be updated periodically, such as monthly, weekly or daily, to provide users with current information regarding the effectiveness of prior recommendations, which may help inspire future users to follow through on the recommendations.

FIG. 5 is a flow chart illustrating a method 300 for retrieving information from a healthcare knowledgebase, such as knowledgebase 140. In the illustrated embodiment, at a first block 305, the user purchases a data acquisition device comprising one or more sensors and potentially some additional conditioning and computing components. As described above, the user may purchase the device from a retailer, via mail order, telephone sales, or over the Internet, without a prescription or other intervention by a physician or any other trained healthcare worker.

The data acquisition device may comprise a wide variety of suitable devices, such as, for example, a home polysomnography device, diabetes monitoring device, drug measurement kit, or home cardiology device, with appropriate sensors or other data acquisition components. For example, in some embodiments, the device comprises a multi-channel home polysomnography device with a plurality of sensors, such as a pulse oximetry sensor, airflow sensor, pressure sensor (e.g., Tygon™ Tube), leg movement sensor, and a microphone. In other embodiments, the device may include other sensors to measure EEG, EKG, or REM signals, or it may comprise a single-use data collection kit used to perform urine analysis or blood analysis.

Once purchased, at a next block 310, the user may register the device. In some embodiments, block 310 involves completing online web forms, mail in registration cards, automated telephone registration, or registration via talking to a customer service representative. During block 310, a device serial number may be associated with a specific user, and the user may also be provided with additional information such as a user survey. In some embodiments, block 310 may also involve additional activities, such as activating the device, updating device drivers, or providing additional user instructions or information.

Once registration is complete, at a next block 315, the device acquires data from the user. For example, in some embodiments, the device comprises a multi-channel home polysomnography device that acquires biometric parameters such as air flow, blood oxygenation, heart rate, snoring or respiratory noises, limb activity, EKG data, and EEG data, for several hours while the user is sleeping. In other embodiments, data might be collected over a period of minutes or seconds. For example, in some cases, the device may take the form of a public kiosk (in which case, the device would not be purchased or registered by a user, as discussed above) that acquires blood pressure data from a user over a period of about 30 seconds.

Once the data is acquired, at a next block 320, the data is transferred to and recorded on a suitable storage medium. The storage medium may be local (e.g., a local hard drive, optical drive or tape drive) or remote (e.g., an Internet-based storage solution). In addition, a wide variety of suitable storage technologies may be employed, such as, for example, electronic, magnetic, optical, pen based or printed storage technologies. The data may be transferred, if needed, using wired or wireless transmission links, together with a variety of suitable telecommunications standards and protocols known to those of ordinary skill in the art.

Once recorded, at a next block 325, the data is analyzed to generate a diagnosis of the user's condition. In some embodiments, the diagnosis comprises graphical or numeric parameters regarding the user's condition, such as, for example, “blood pressure greater that 130/85” or “O₂ saturation less than 95%” or “Apnea+Hyponea Index (AHI) greater than 17.” The diagnosis may also comprise a textual description of the user's condition, such as, for example, “obstructed sleep apnea” or “hypertension” or “type 1 diabetes.”

At a next block 330, the diagnosis is used to query a knowledgebase, such as knowledgebase 140 described above. As discussed earlier, this query may comprise a simple table look up to a flat file database or include structured relational databases, fuzzy logic, complex algorithms, and artificial intelligence. At a next block 335, the user is provided with recommendations, which are customized to the user's unique condition, as determined by the biometric sensors. In some embodiments, as shown in FIG. 5, the recommendations may include professional referrals 145A, published articles 145B, recommended websites 145C, or recommended behaviors 145D.

A professional referral 145A may comprise a general suggestion that a user seek professional medical assistance, or a more specific recommendation, such as, for example, a referral to a physician with a particular specialty (e.g., sleep disorders) or even a referral to a specific physician or other healthcare provider. Examples of published articles 145B may include articles on general health and wellness topics, or more detailed articles on specific topics published in peer-reviewed medical journals, such as the Journal of American Medical Association (JAMA).

Recommended websites 145C may include general health and wellness websites, such as, for example, www.WebMD.com or www.emedicine.com. Recommended websites 145C may also include more specialized websites, such as, for example, www.apneasupport.org, or customized websites with pre-screened content developed and/or selected by one or more experts in a given specialty. Recommended behaviors 145D may include a variety of recommendations, such as, for example, increasing exercise, reducing alcohol consumption, undergoing physical therapy, or implementing diet restrictions. In some embodiments, the recommendations provided at block 335 result in a user-specific treatment plan and targeted educational materials that advantageously provide better health, more informed users, and increased well-being.

Although this invention has been described in terms of certain preferred embodiments, other embodiments that are apparent to those of ordinary skill in the art, including embodiments that do not provide all of the features and advantages set forth herein, are also within the scope of this invention. Therefore, the scope of the present invention is defined only by reference to the appended claims and equivalents thereof.

What is claimed is:

1. A method for automatically retrieving information from a computerized healthcare knowledgebase, the method comprising:
collecting data about a user from one or more biometric sensors;
analyzing the data to determine a condition of the user;
processing the user condition information to formulate a query of the computerized healthcare knowledgebase for relevant content; and
presenting the relevant content to the user in an automated fashion.

2. The method of claim 1, wherein processing the user condition information includes utilizing Boolean logic, complex algorithms, fuzzy logic, or artificial intelligence.

3. The method of claim 1, wherein collecting data comprises measuring pulmonary functions, performing blood analysis, performing urine analysis, recording snoring, recording respiratory noises, or measuring limb activity.

4. The method of claim 1, wherein the user is sleeping while the biometric sensors are collecting data.

5. The method of claim 1, further comprising administering a user survey requesting user behavior, blood sugar level, diet, previous medical history, or family history.

6. The method of claim 1, wherein the user may purchase the data collecting device comprising biometric sensors at a retailer, via mail order, telephone sales, or over the Internet.

7. A healthcare knowledgebase having a plurality of records, each record comprising:
a reference to healthcare related multimedia content; and
numeric biometric limit parameters indicating one or more physiological conditions to which the referenced content is relevant,
wherein the numeric biometric limit parameters comprise rolling statistical information that is updated periodically based on data collected from a plurality of users over time.

8. The healthcare knowledgebase of claim 7, wherein the records are organized into a relational database, a table, a flat file data database, structured data tables, relational data sets, or free form data.

9. The healthcare knowledgebase of claim 7, wherein the knowledgebase includes links to internet websites, multimedia content, published articles, journal articles, relevant videos, audio podcasts, video podcasts, audio content, animations, photographs or targeted professional referrals and recommendations.

10. The healthcare knowledgebase of claim 7, wherein the knowledgebase is stored on a local hard drive, an optical drive, a tape drive, or a remote Internet-based storage solution.

11. The healthcare knowledgebase of claim 7, wherein the numeric biometric limit parameters include a temporal oximetry tracing, a time versus O₂ saturation graph, a heart rate summary table, a SaO₂ summary table, or a respiratory events table.

12. An automated system for presenting healthcare information to a patient, the system comprising:
a patient data module with one or more biometric sensors for collecting data from the patient;
a compute module in communication with the patient data module, the compute module being configured to analyze the data collected by the biometric sensors by referencing a symptom database;
a query module in communication with the compute module, the query module being configured to query a healthcare knowledgebase for information relevant to a condition of the patient, based on the data collected by the biometric sensors,
wherein the healthcare knowledgebase comprises a plurality of multimedia presentations and articles.

13. The system of claim 12, wherein the biometric sensors include an oxygen saturation sensor, EKGs, EEGs, blood pressure sensors, motion sensors, photodiodes, light sensors, microphones, REM sensors, respiratory monitors, glucose sensors, or blood analysis devices.

14. The system of claim 12, wherein the biometric sensors comprise disposable sensors.

15. The system of claim 12, wherein the compute module communicates with the patient module via the Internet.

16. The system of claim 12, wherein the patient module, compute module, query module, symptom database, and healthcare knowledgebase are contained within a single physical unit.

17. The system of claim 12, wherein the query module comprises algorithms, software, or hardware to query the healthcare knowledgebase.

18. The system of claim 12, wherein the system includes a home polysomnography device.

19. The system of claim 12, wherein the compute module comprises a desktop computer, a notebook computer, tablet computer, handheld computer, PDA, or mobile phone.

20. The system of claim 12, wherein the patient data module is assigned a unique serial number.
